(19) United States
(12) Patent Application Publication
(10) Pub. No.: US 2020/0197516 A1
(43) Pub. Date: Jun. 25, 2020

(54) Antagonists of Products of the HS.459642 Unigene Cluster for the Inhibition of Proliferation, Development or Differentiation of Stem Cells Including Cancer Stem Cells

(71) Applicant: Lloyd S. Gray, Louisa, VA (US)

(72) Inventor: Lloyd S. Gray, Louisa, VA (US)

(73) Assignee: Cavion, Inc., Charlottesville, VA (US)

(21) Appl. No.: 16/685,279

(22) Filed: Nov. 15, 2019

Related U.S. Application Data
(63) Continuation of application No. 13/609,942, filed on Sep. 11, 2012, now abandoned.
(60) Provisional application No. 61/533,545, filed on Sep. 12, 2011.

(51) Int. Cl. A61K 39/395; A61K 31/4184; C12N 15/113; A61K 45/06; A61K 31/496

(52) U.S. Cl. CPC A61K 39/395; A61K 31/4184; C12N 15/113; C12N 2310/11; C12N 15/1138; A61K 31/496; A61K 45/06

(57) ABSTRACT

The present disclosure provides methods and compositions for inhibiting the proliferation, differentiation, or development of stem cells and cancer stem cells in a patient in need thereof. The methods involve administering to a patient a therapeutically effective amount of an antagonist of an Hs.459642 Unigene Cluster product, such as an inhibitor of CACNA1H. The compositions include an antagonist of an Hs.459642 Unigene Cluster product, such as an inhibitor of CACNA1H. Specific antagonists such as antibodies and antisense oligonucleotides, and combination therapy with one or more additional anti-cancer agents, are also provided by this disclosure. Such methods, antagonists, and compositions can be useful, for example, in the treatment of cancer.

### BACKGROUND OF THE DISCLOSURE

Stem cells are undifferentiated cells that have extensive proliferation potential, can differentiate into several cell lineages, and repopulate tissues upon transplantation. Stem cells can give rise to more progenitor cells having the ability to generate a large number of mother cells that can in turn give rise to differentiated, or differentiable daughter cells. The quintessential stem cell is the embryonic stem cell, as it has unlimited self-renewal and pluripotent differentiation potential (Orkin, Int. J. Dev. Biol. 42:927-34, 1998; Reubinoff et al., Nat Biotech. 18:399404, 2000; Shamblott et al., Proc. Natl. Acad. Sci. U.S.A. 95:13726-31, 1998; Thomson et al., Science 282:114-7, 1998; Thomson et al., Proc. Natl. Acad. Sci. USA. 92:7844-8, 1995; Williams et al., Nature 336:684-7, 1988).

Like stem cells, cancer cells have extensive proliferation capacity. However, in contrast to stem cells, which respond to external factors in a controlled manner, cancer cells do not respond appropriately to external factors, and display uncontrolled proliferation. Recent findings indicate that tumor formation and growth are driven by a subpopulation of cancer cells, termed cancer stem cells (CSCs), which have the capacity to seed and generate primary and secondary tumors. Studies have uncovered numerous similarities between CSCs and embryonic stem cells, including genetic regulation of self-renewal and pluripotency by microRNA (Mallick B, et al., RNA Biol 8:3, 2011). Evidence in several types of cancer shows that pathways prominent in normal stem cell function, such as Wnt, Notch, Shh (sonic hedgehog), and XIAP (X-linked inhibitor of apoptosis protein) become dysregulated in CSCs (Reya et al., Nature 414:105, 2001; Dontu G et al., Breast Cancer Res 6:R605, 2004; Rosner A K et al., Proc. Natl. Acad. Sci. U.S.A. 100:15853; Yang LZ et al., Cancer Res 63:6815, 2003; Liu S et al., Breast Cancer Res 7:86, 2005; Mikaelian I et al., Breast Cancer Res 6:R668, 2004).

CSCs are also referred to as tumor initiating cells, cancer stem-like cells, cancer stem cell-like cells, highly tumorigenic cells, or super malignant cells. A cancer stem cell can be the founder cell of a tumor (i.e., it is the progenitor of the cancer cells that comprise the tumor bulk). CSCs are distinguished from other tumor cells by their ability to successfully seed new tumors when implanted in low numbers into experimental animals. In contrast, non-CSC cells cannot initiate tumor growth in vivo even when implanted in high numbers (Dalerba et al., Annu. Rev. Med. 58:267-284, 2007). Thus, CSCs contribute significantly to carcinogenesis, cancer metastasis, and cancer reoccurrence.

Cancer stem cells comprise a unique subpopulation (often 0.1-10% or so, but as much as 0.1 to 20% or more) of a tumor that, relative to the remaining 90% or so of the tumor (i.e., the tumor bulk), are more tumorigenic, relatively more slow-growing or quiescent, and often relatively more chemoresistant than the tumor bulk. Given that conventional therapies and regimens are typically designed to attack rapidly proliferating cells (i.e. those cancer cells that comprise the tumor bulk), cancer stem cells are therefore more resistant than faster growing tumor cells to conventional therapies and regimens. Cancer stem cells can express other features which also make them more chemoresistant, such as multi-drug resistance, higher DNA repair capability, and elevated anti-apoptotic activity. These attributes constitute a key reason for the failure of standard oncology treatment regimens to ensure long-term benefit in most patients with advanced stage cancers, i.e., the failure to adequately target and eradicate cancer stem cells.

The efficacy of cancer treatments is often measured by the amount of tumor mass they kill off. As CSCs generally form a very small proportion of the tumor and have markedly different biologic characteristics than their differentiated progeny, the measurement of tumor mass may not necessarily select for drugs that act specifically on the stem cells. In other words, conventional chemotherapies kill differentiated or differentiating cells, which form the bulk of the tumor that are unable to generate new cells. The population of cancer stem cells which gave rise to the tumor could remain untouched and cause a relapse of the disease. Furthermore, treatment with chemotherapeutic agents may only leave chemotherapy-resistant cancer stem cells, so that the ensuing tumor will most likely also be resistant to chemotherapy and radiotherapy.

Since surviving cancer stem cells can repopulate the tumor and cause relapse, it would be possible to treat patients with aggressive, non-resectable tumors and refractory or recurrent cancers, as well as prevent the tumor metastasis and recurrence, by targeting cancer stem cells. Treatments targeted to cancer stem cells and cancer stem-like cells show promise as cancer therapies. Development of specific therapies targeted at cancer stem cells therefore holds hope for improvement of survival and quality of life of cancer patients, especially for sufferers of metastatic disease. The high rates of recurrence and metastasis, even following surgery, chemotherapy, radiation, small molecule and antibody therapies—all of which shrink tumors but do not eliminate immortal tumor cells—underscore the need to identify new therapeutic strategies that specifically target and kill cancer stem cells in order to eliminate recurrence and metastatic disease.

### BRIEF SUMMARY OF THE DISCLOSURE

The present disclosure provides methods and pharmacologic compositions for inhibiting the proliferation, differentiation, or development of stem cells and cancer stem cells. The compositions include an antagonist of an Hs.459642 Unigene Cluster product. The methods involve administering to a patient a therapeutically effective amount of an antagonist of an Hs.459642 Unigene Cluster product. In a preferred embodiment, the antagonist is an inhibitor of the Hs.459642 Unigene Cluster product CACNA1H. Specific antagonists, such as antibodies and antisense oligonucleotides, are also provided by this disclosure. Such methods, antagonists, and compositions can be useful, for example, in the treatment of cancer.

Combination therapy with an additional anti-cancer agent is also provided by this disclosure. The additional anti-cancer therapy can include, but is not limited to, surgery, radiation therapy, or administration of a chemotherapeutic agent. Combination therapy can be an interlaced therapy, where the compositions of the invention are provided to a patient, followed by a subsequent anti-cancer therapy. Alternatively, combination therapy can involve simultaneous, or overlapping, administration of a composition of the invention with a further anti-cancer therapy.

The antagonist of an Hs.459642 Unigene Cluster product can be a Hs.459642 Unigene Cluster product-specific antibody. The antibody can be polyclonal or monoclonal, and can be fully or partially humanized. In a particular example, the antibody is a CACNA1H-specific antibody. The antagonist can also be an antisense oligonucleotide molecule that inhibits a Hs.459642 Unigene Cluster product such as CACNA1H.

### BRIEF DESCRIPTION OF THE FIGURES

FIG. 1. Developmental expression of CACNA1H. CACNA1H is expressed in embryoid body, blastocyst, fetus, juvenile, and adult tissues. The far right column displays the number of CACNA1H ESTs in the numerator with the total number of ESTs in the pool given as the denominator. The middle column is the number of CACNA1H tags normalized to a pool size of 1,000,000.

FIG. 2. Developmental expression of CACNA1G. CACNA1G is expressed in fetus and adult tissues. The far right column displays the number of CACNA1G ESTs in the numerator with the total number of ESTs in the pool given as the denominator. The middle column is the number of CACNA1G tags normalized to a pool size of 1,000,000.

FIG. 3. Developmental expression of CACNA1I. CACNA1I is expressed in blastocyst, fetus, juvenile, and adult tissues. The far right column displays the number of CACNA1I ESTs in the numerator with the total number of ESTs in the pool given as the denominator. The middle column is the number of CACNA1I tags normalized to a pool size of 1,000,000.

FIG. 4. Effect of mibefradil (Mi) on sensitizing glioma stem cells (GSC) to Temozolomide (TMZ). The GSCs were seeded in 24 well plates as 50,000 cells/well overnight. They were grown in serum-free neurobasal media (Invitrogen), supplemented with human recombinant EGF and bFGF (R&D Systems) at 37° C. in 5% CO₂, 95% O₂. Cells were treated with the T type calcium channel blocker mibefradil (30 nM) for 24 hrs, washed and then treated with Temozolomide (5 μM) for 24 hrs The reagent alamarBlue® (Invitrogen) was added to each well to arrive at a final concentration of 10% alamarBlue® in each well and fluorescence intensity was determined according to the manufacturers instructions. Plates were incubated for 1-2 hrs and 100 μl of supernatant was transferred to a 96 well plate for alamarBlue® reading.

FIG. 5. Effect of TTL-1177 (TTL) on sensitizing GSCs to Temozolomide (TMZ). The GSCs were seeded using the same method as in FIG. 4. Cells were treated with the T type calcium channel blocker TTL-1177 (30 μM) for 24 hrs and then treated with Temozolomide (5 μM) for 24 hrs. The cells were tested using an alamarBlue® assay as described in FIG. 4.

### DETAILED DESCRIPTION OF THE DISCLOSURE

This disclosure presents treatments for cancer that target cancer stem cells (CSCs) and cancer stem-like cells by inhibition of the products of the Hs.459642 Unigene Cluster. This disclosure further presents methods of treating cancer comprising administration of combination therapy to target both CSC and non-CSC cells in a tumor, thus treating multiple cancer cell types and creating more effective treatment regimes. Inhibition of products of the Hs.459642 Unigene Cluster is useful for preventing proliferation, growth, and development of cancer cells and all types of stem cells, including but not limited to embryonic stem cells, adult stem cells, cancer stem cells, and cancer stem-like cells.

The present invention is related to the discovery that a product or products of the Hs.459642 Unigene Cluster are present in CSCs. The invention provides a method of inhibiting the growth of tumors that express CSCs. The method comprises administering to an individual a composition comprising an inhibitor of a product of the Hs.459642 Unigene Cluster in an amount effective to inhibit the growth of the CSCs. Also provided is a method for inhibiting metastasis of cancer cells in an individual, wherein the cancer cells comprise one or more products of the Hs.459642 Unigene Cluster. The method comprises administering to the individual an amount of an agent comprising an inhibitor of a product of the Hs.459642 Unigene Cluster effective to inhibit the metastasis.

The Homo sapien (Hs.) 459642 Unigene Cluster is located on human chromosome 16. A “unigene cluster” represents a set of transcript sequences oriented within the same transcription locus, the transcription locus being a gene or expressed pseudogene. Hs.459642 comprises at least the CaV3.2/CACNA1H (calcium channel, voltage-dependent, T type, alpha 1H subunit) and its splice and transcript variants and the CaV3.2 proteins with Genbank Accession Nos. NP_006921.2 and NM_001005407.1, among others. A “Hs.459642 Unigene Cluster product” or simply, a “product” as recited herein, refers to anything produced or expressed from the Hs.459642 Unigene Cluster locus, including complete or partial nucleotide transcripts, complete or partially translated peptides and immature and mature proteins, and fragments thereof

A preferred Hs.459642 Unigene Cluster product is the CACNA1H calcium channel. This Hs.459642 product is an H isoform T type calcium channel. The involvement of CaV3.2/CACNA1H in proliferation and differentiation has been described previously (Lory P et al., Cell Calcium 40:135-46, 2006).

T-type calcium/Ca²⁺ channels are low voltage activated ion channels that open following small membrane depolarizations. There are three T-type α1 calcium channel subunits: α_{1G}, α_{1H}, and all. The Hs.459642 UniGene cluster contains the sequence for canonical α_{1H}. α₁ is the channel's ion conducting protein, which comprises four domains with each domain comprising six transmembrane segments. α1 subunits of T-type calcium channels can function as stand-alone complexes, unlike other α1 calcium channel subunits. T-type Ca²⁺ channels have been primarily studied in the context of neuronal and cardiomyocyte function, and have been implicated in hyperexcitability disorders, such as epilepsy and cardiac dysfunction. Voltage gated calcium channels are not generally expressed in non-excitable cells, but there is evidence that T-type calcium channels are expressed in cancer cells of non-excitable lineages (Taylor J T et al., World J Gastroenterol. 14:4984-4991, 2008).

T-type Ca²⁺ channels are activated and inactivated by small membrane depolarizations, and display slow deactivation rates. Thus, these channels can carry depolarizing current at low membrane potentials and mediate cellular “window” currents, which occur within the voltage overlap between activation and steady state inactivation at low or resting membrane potentials (Tsien R W, et al. In: Low-voltage-activated T-type Ca2+ channels, Chester: Adis International Ltd, pp. 1-394, 1998; Crunelli V, et al., J Physiol. 562:121-129, 2005). T-type Ca²⁺ channels can maintain window current at non-stimulated or resting membrane potentials, thereby allowing a sustained inward calcium current carried by a portion of channels that are not inactivated (Bean B P, McDonough S I, Neuron 20:825-828, 1998). Mediation of window current allows T-type Ca²⁺ channels to regulate intracellular calcium levels, both in electrically firing cells such as neurons, and in non-excitable tissues, under non-stimulated or resting cellular conditions.

Intracellular calcium regulation is an important element of multiple signaling pathways regulating cell cycle transition and apoptosis. Cancer cells are able to progress through the cell cycle and bypass normal calcium-mediated checkpoints, indicating that cancer cells have developed alternative mechanisms to regulate intracellular calcium. New evidence that cancer cells express T-type calcium channels suggests that these channels play a role in checkpoint-independent cell cycle progression and cellular proliferation (Taylor J T et al., World J Gastroenterol. 14:4984-4991, 2008). However, previous studies linking calcium signaling and expression of T-type calcium channels have not identified the presence of these unique calcium channels in CSC subpopulations, nor is such an assumption implicit in the finding that some cancer cells express T-type calcium channels. In fact, the correlation between calcium and cell proliferation suggests that T-type calcium channels would be present in fast-dividing, rapidly proliferating non-stem cancer cells, rather than in cancer stem cells.

The inventor has discovered that the CACNA1H T-type calcium channel is expressed by CSCs, and that treatments designed to prevent expression and/or activity of products of the Hs.459642 Unigene Cluster, particularly treatments that prevent expression and/or activity of CACNA1H and products of the CACNA1H locus, can be used to target cancer stem cells as well as non-stem cancer cells, thus treating a more complete spectrum of cancerous cells. Inhibition of products of the Hs.459642 Unigene Cluster, particularly inhibition of CACNA1H and CACNA1H products, can block cancer cell proliferation and also prevent CSC activity, thus reducing tumor size and growth and also preventing malignancy, metastasis, and tumor recurrence and relapse. Inhibition of products of the Hs.459642 Unigene Cluster can also be useful for prevention of undesirable proliferation and/or activity of non-cancerous stem cells, such as embryonic or adult stem cell proliferation and/or activity.

The developmental expression and relative abundance of Expressed Sequence Tags (ESTs) of CACNA1H is shown in FIG. 1. FIG. 1 identifies CACNA1H expression in the embryoid body, blastocyst, and fetus, as well as in juvenile and adult tissues, while expression is absent in neonates and infants. Normalized to a pool size of 1 million ESTs, expression of CACNA1H is evident at highly elevated levels in embryoid bodies (approximating 56 tags per million, more than double the expression of this gene at any other developmental stage). Embryoid bodies are aggregates of embryonic stem cells, and the abundant expression of CACNA1H indicates a high level of Hs.459642 Unigene Cluster activity in these cells. High expression levels in stem cells correlates with expression of the same product(s) in CSCs, indicating that CACNA1H is active in cancer stem cells as well.

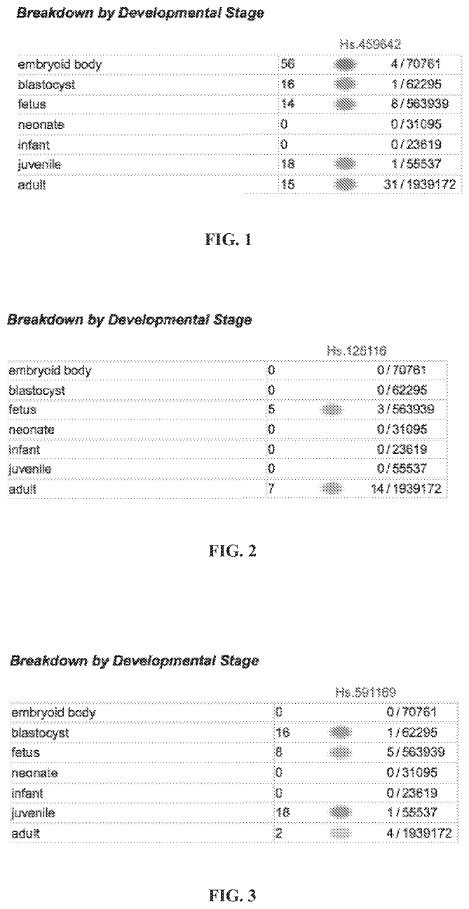

The abundant expression of CACNA1H ESTs in the embryoid body is not a non-specific trait of all T type calcium channel isoforms, because neither CACNA1G nor CACNA1I ESTs are expressed in embryoid bodies (FIGS. 2 and 3). Thus, CACNA1H is uniquely important for stem cell activity, and cancer stem cell activity, relative to other T type calcium channels.

The term “stem cell” as used herein refers to a cell that is capable of differentiating into a number of final, differentiated cell types. Stem cells may be totipotent, pluripotent, or unipotent cells. Totipotent stem cells typically have the capacity to develop into any cell type and are usually embryonic in origin. Pluripotent cells are typically cells in a stem cell line capable of differentiating into several different, final differentiated cell types. Unipotent cells are typically capable of differentiating into a single cell type. Non-embryonic stem cells are usually pluripotent or unipotent. Unipotent and pluripotent stem cells can originate from various tissue or organ systems, including, but not limited to, blood, nerve, muscle, skin, gut, bone, kidney, liver, pancreas, thymus, and the like.

As used herein, a “tumor” cell or “cancer” cell means an abnormal cell exhibiting uncontrolled proliferation and potential to invade surrounding tissues.

As used herein, the term “cancer stem cell” or “CSC” or “cancer stem-like cell” or “CSLC” refers to a cell that can be a progenitor of, or give rise to a progenitor of, a highly proliferative cancer cell. A cancer stem cell has the ability to re-grow a tumor as demonstrated by its ability to form tumors in immuno-compromised mammal such as mice, and typically to form tumors upon subsequent serial transplantation in immuno-compromised mammals such as mice. Cancer stem cells are also typically slow-growing relative to the bulk of a tumor; that is, cancer stem cells are generally quiescent. A cancer stem cell may represent approximately 0.1 to 20% of a tumor. A cancer stem-like cell is cancer cell that displays some or all of the characteristics of a cancer stem cell.

CSCs can show one or more activities characteristic of embryonic stem cells, such as loss of contact inhibition, anchorage independent growth, de novo expression of alkaline phosphatase and activation of the germ line specific Oct4 promoter. Oct4, a member of the Pou domain, class 5, transcription factors (Pou 5fl) (Genbank Accession No. 568053) is one of the mammalian POU transcription factors expressed by early embryo cells and germ cells, and is a marker for pluripotent stem cells in mammals.

The term “progenitor” as used herein, refers to a cell that is committed to a particular cell lineage and which gives rise to cells of this lineage by a series of cell divisions. A progenitor cell can more differentiated than a stem cell but is not itself fully differentiated. An example of a progenitor cell would be a myoblast, which is capable of differentiation to only one type of cell, but is itself not fully mature or fully differentiated.

The terms “proliferation” and “growth” as used interchangeably herein with reference to cells, refer to an increase in the number of cells of the same type by cell division, rapid and repeated cellular reproduction, cell cycling, and cell growth, particularly uncontrolled cellular growth. “Development” refers to the progression from a smaller, less complex, or benign form to a larger, more complex, or neoplastic form. For example, a tumor may develop from a small mass to a larger mass. Cancer stem cell development can refer to the progression from a non-cancerous cell state to a cancerous cell state, or the progression from non-neoplastic tissue formation to neoplastic or tumor formation.

The term “differentiation” as used herein, refers to a developmental process whereby cells become specialized for a particular function, for example, where cells acquire one or more morphological characteristics and/or functions different from that of the initial cell type. The term “differentiation” includes both lineage commitment and terminal differentiation processes. Differentiation may assessed, for example, by monitoring the presence or absence of lineage markers, using immunohistochemistry or other procedures known to a worker skilled in the art. Differentiated progeny cells derived from progenitor cells may be, but are not necessarily, related to the same germ layer or tissue as the source tissue of the progenitor cells.

The term “terminal differentiation” as used herein, refers to the final differentiation of a cell into a mature, fully differentiated cell. Usually, terminal differentiation is associated with—withdrawal from the cell cycle and cessation of proliferation. The terms “lineage commitment” and “specification,” as used interchangeably herein, refer to the process a stem cell undergoes in which the stem cell gives rise to a progenitor cell committed to forming a particular limited range of differentiated cell types. Committed progenitor cells are often capable of self-renewal or cell division.

The present invention provides methods of inhibiting proliferation, growth, and development, and/or inducing terminal differentiation of cancer stem cells, by contacting the cells, directly or indirectly, with one or more inhibitors of products of the Hs.459642 Unigene Cluster, particularly inhibitors of CACNA1H. These methods are also useful for inhibiting proliferation, growth, and development, and/or inducing terminal differentiation of non-cancer stem cells such as embryonic or adult stem cells.

“Inhibition” as used herein refers to reduction or prevention of activity.

Activities of a product or products of the Hs.459642 Unigene Cluster which can be inhibited by the present invention include, but are not limited to: cellular calcium uptake; regulation and/or mediation of intracellular calcium levels; regulation and/or mediation of intracellular window currents; calcium-mediated signaling and/or regulation of calcium signaling pathways; initiating and/or maintaining cellular growth and proliferation, particularly excessive or unwanted proliferation; initiating and/or maintaining neoplasia and/or tumor growth; and initiating and/or maintaining angiogenesis and/or metastasis.

An “antagonist” inhibits activity or function. For example, a compound can act as an antagonist by inhibiting, reducing or eliminating protein expression, or preventing protein activity, or preventing interaction of protein with other proteins, resulting in an inhibition of a protein-mediated function or signaling. Examples of antagonists include peptides, polypeptides, proteins, antibodies, antisense oligonucleotides, RNAi/siRNA, small molecules, chemotherapeutic agents, and fragments, derivatives and analogs thereof, that inhibit the activity or function of one or more products of the Hs.459642 Unigene Cluster, and particularly that inhibit the activity or function of CACNA1H.

The terms “peptide”, “polypeptide”, and “protein”, as used herein, refer to a sequence of amino acid residues linked together by peptide bonds or modified peptide bonds. Typically, a polypeptide or protein is at least six amino acids long and a peptide is at least 3 amino acids long. The protein, polypeptide or peptide can be naturally occurring, recombinant, synthetic, or a combination of these. The protein, polypeptide or peptide can be a fragment of a naturally occurring protein or polypeptide. The terms polypeptide and peptide also encompass peptide analogues, peptide derivatives and peptidomimetic compounds. Such compounds are well known in the art and may have significant advantages over naturally occurring peptides, including, for example, greater chemical stability, increased resistance to proteolytic degradation, enhanced pharmacological properties (such as, half-life, absorption, potency and efficacy), altered specificity (for example, a broad-spectrum of biological activities) and/or reduced antigenicity.

A “variant” protein, polypeptide, peptide, or fragment thereof, is one in which one or more amino acid residues has been deleted, added or substituted for those that appear in the amino acid sequence of the wild-type protein. In the context of the present invention, a variant also retains substantially the same activity as the wild-type protein. Typically, when a variant contains one or more amino acid substitutions they are “conservative” substitutions. A conservative substitution involves the replacement of one amino acid residue by another residue having similar side chain properties. As is known in the art, the twenty naturally occurring amino acids can be grouped according to the physicochemical properties of their side chains. Suitable groupings include alanine, valine, leucine, isoleucine, proline, methionine, phenylalanine and tryptophan (hydrophobic side chains); glycine, serine, threonine, cysteine, tyrosine, asparagine, and glutamine (polar, uncharged side chains); aspartic acid and glutamic acid (acidic side chains) and lysine, arginine and histidine (basic side chains). Another grouping of amino acids is phenylalanine, tryptophan, and tyrosine (aromatic side chains). A conservative substitution involves the substitution of an amino acid with another amino acid from the same group.

Protein, polypeptide and peptide inhibitors contemplated by the present invention include proteins that naturally inhibit a product of the Hs.459642 Unigene Cluster, and active fragments and variants of such inhibitors. Other examples of inhibitors include peptide derivatives, analogues or peptidomimetics that inhibit a product of the Hs.459642 Unigene Cluster and thereby prevent calcium-activated signaling activity. In a particular example, the inhibitor inhibits CACNA1H.

The present invention also contemplates the use of biologically inactive proteins or fragments of proteins that interfere with the action of the wild-type protein and thus, act as inhibitors of protein activity. Examples include dominant negative mutants. Biologically inactive proteins or fragments contemplated by the present invention are those that have substantially less activity than the wild-type protein. Candidate inhibitory fragments can be selected from random fragments generated from the wild-type protein. Methods for generating the candidate polypeptide fragments are well known to workers skilled in the art. Biologically inactive proteins can also be generated, for example, by site-directed or random mutagenesis techniques of nucleic acids encoding the protein, or by inactivation of the protein by chemical or physical means.

In addition to inhibition of proliferation, differentiation or development of cancer stem cells, inhibitors of the products of the Hs.459642 Unigene Cluster can inhibit proliferation, differentiation or development of embryonic stem cells, adult stem cells, and other pluripotent, multipotent, or unipotent stem cells as well.

Cancer or a neoplastic disease, including, but not limited to, neoplasms, tumors, metastases, leukemias or any disease or disorder characterized by uncontrolled cell growth, can be prevented, treated, and/or managed by administering to a subject in need thereof a prophylactically or therapeutically effective amount of an inhibitor of a product of the Hs.459642 Unigene Cluster, particularly an inhibitor of CACNA1H.

Any type of cancer can be prevented, treated and/or managed in accordance with the invention. Non-limiting examples of cancers that can be prevented, treated and/or managed in accordance with the invention include: leukemias; lymphomas; multiple myelomas; bone and connective tissue sarcomas; brain tumors; breast cancer; adrenal cancer; thyroid cancer; pancreatic cancer; pituitary cancers; eye cancers; vaginal cancers; cervical cancers; uterine cancers; ovarian cancers; esophageal cancers; stomach cancers; colon cancers; rectal cancers; liver cancers; gallbladder cancers; cholangiocarcinomas; lung cancers; testicular cancers; prostate cancers; penile cancers; oral cancers; basal cancers; salivary gland cancers; pharynx cancers; skin cancers; kidney cancers; Wilms' tumor; bladder cancers.

As used herein, the terms “subject” and “patient” are used interchangeably and refer to an animal, preferably a mammal such as a non-primate (e.g., cows, pigs, horses, cats, dogs, rats etc.) and a primate (e.g., monkey and human), and most preferably a human.

As used herein, “treatment” refers to clinical intervention in an attempt to alter the disease course of the individual or cell being treated, and can be performed either for prophylaxis or during the course of clinical pathology. Therapeutic effects of treatment include without limitation, preventing occurrence or recurrence of disease, alleviation of symptoms, diminishment of any direct or indirect pathological consequences of the disease, decreasing the rate of disease progression, amelioration or palliation of the disease state, and remission or improved prognosis. For example, treatment of a cancer patient may be reduction of tumor size, elimination of malignant cells, prevention of metastasis, or the prevention of relapse in a patient whose tumor has regressed.

As used herein, the terms “therapeutically effective amount” and “effective amount” are used interchangeably to refer to an amount of a composition of the invention that is sufficient to result in the prevention of the development, recurrence, or onset of cancer stem cells or cancer and one or more symptoms thereof, to enhance or improve the prophylactic effect(s) of another therapy, reduce the severity and duration of cancer, ameliorate one or more symptoms of cancer, prevent the advancement of cancer, cause regression of cancer, and/or enhance or improve the therapeutic effect(s) of additional anticancer treatment(s).

A therapeutically effective amount can be administered to a patient in one or more doses sufficient to palliate, ameliorate, stabilize, reverse or slow the progression of the disease, or otherwise reduce the pathological consequences of the disease, or reduce the symptoms of the disease. The amelioration or reduction need not be permanent, but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

In certain embodiments of the invention, the therapeutically effective amount is an amount that is effective to achieve one, two or three or more of the following results once it is administered: (1) a reduction or elimination of the cancer stem cell population; (2) a reduction or elimination in the total cancer cell population; (3) a reduction in the growth or proliferation of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) the size of the tumor is maintained and does not increase or increases by less than 10%, or less than 5%, or less than 4%, or less than 2%, (10) an increase in the number of patients in remission, (11) an increase in the length or duration of remission, (12) a decrease in the recurrence rate of cancer, (13) an increase in the time to recurrence of cancer, (14) an amelioration of cancer-related symptoms and/or quality of life and (15) a reduction in drug resistance of the cancer cells.

In some embodiments, the amount or regimen of an inhibitor of a product of the Hs.459642 Unigene Cluster results in a reduction in the bulk tumor size as well as a reduction in the cancer stem cell population. In certain embodiments, the reduction in the bulk tumor size; the reduction in the bulk tumor size and the reduction in the cancer stem cell population, including drug resistant cancer stem cells; or the reduction in the bulk tumor size, the reduction in the cancer stem cell population and the reduction in the cancer cell population are monitored periodically. Accordingly, in one example, the invention provides a method of preventing, treating and/or managing cancer in a subject, the method comprising: (a) administering to a subject in need thereof one or more doses of an effective amount of an inhibitor of a product of the Hs.459642 Unigene Cluster. In a particular example, the inhibitor inhibits CACNA1H.

In accordance with the present invention, there are provided antibodies that antagonize products of the Hs.459642 Unigene Cluster to inhibit proliferation, differentiation or development of stem cells including those involved in cancer. Such antibodies can be polyclonal or monoclonal and generated in any suitable species. Monoclonal antibodies may be native to the generating species or fully or partially humanized. In a particular example, the antibody or antibodies inhibit CACNA1H.

Various methods for the preparation of antibodies are known in the art (see, Antibodies: A Laboratory Manual, CSH Press, Eds., Harlow, and Lane (1988); Harlow, Antibodies, Cold Spring Harbor Press, N.Y. (1989)). For example, antibodies can be prepared by immunizing a suitable mammalian host with a sample of whole cells isolated from a patient. Briefly, such methods of generating an immune response (e.g. humoral and/or cell-mediated) in a mammal, comprise the steps of: exposing the mammal's immune system to a Hs.459642 Unigene Cluster product or products, such as CACNA1H, so that the mammal generates an immune response that is specific for the Hs.459642 Unigene Cluster product or products (e.g. generates antibodies that specifically recognize one or more Hs.459642 Unigene Cluster product protein epitopes).

Antibodies can be produced by cell culture techniques, including the generation of monoclonal antibodies as described herein, or via transfection of antibody genes into suitable bacterial or mammalian cell hosts, in order to allow for the production of recombinant antibodies. In one technique, a sample of a Hs.459642 Unigene Cluster product is initially injected into any of a wide variety of mammals (e.g., mice, rats, rabbits, sheep or goats). A superior immune response may be elicited if the sample is injected along with a carrier protein, such as bovine serum albumin or keyhole limpet hemocyanin. The sample is injected into the animal host, preferably according to a predetermined schedule incorporating one or more booster immunizations, and the animals are bled periodically so that titers of antibodies can be taken to determine adequacy of antibody formation. Polyclonal antibodies specific for the polypeptide may then be purified from such antisera by, for example, affinity chromatography using cells from the patient sample coupled to a suitable solid support.

A “monoclonal antibody” is an antibody obtained from a population of substantially homogeneous antibodies, i.e., the antibodies comprising the population are identical except for possible naturally occurring mutations that are present in minor amounts. Monoclonal antibodies specific for Hs.459642 Unigene Cluster products may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with Hs.459642 Unigene Cluster products). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against Hs.459642 Unigene Cluster products. Hybridomas having high reactivity and specificity for Hs.459642 Unigene Cluster products are important for therapeutic purposes. When the appropriate immortalized cell culture is identified, the cells can be expanded and antibodies produced.

In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction.

The antibodies of the invention can also be produced by recombinant means. Antibodies that bind specifically to Hs.459642 Unigene Cluster products can also be produced in the context of chimeric or complementarity-determining region grafted antibodies of multiple species origin. “Humanized” or human antibodies can also be produced, and are preferred for use in therapeutic contexts. Methods for humanizing murine and other non-human antibodies, by substituting one or more of the non-human antibody sequences for corresponding human antibody sequences, are well known [see for example, Jones et al., Nature 321: 522-525 (1986); Riechmann et al., Nature 332: 323-327 (1988); Verhoeyen et al., Science 239: 1534-1536 (1988), Carter et al., Proc. Natl. Acad. Sci. USA 89: 4285(1993); and Sims et al., J. Immunol. 151: 2296 (1993)]. These humanized antibodies are designed to minimize unwanted immunological response toward rodent antihuman antibody molecules which limits the duration and effectiveness of therapeutic applications of those moieties in human recipients. Accordingly, preferred antibodies used in the therapeutic methods of the invention are those that are either fully human or humanized and that bind specifically to Hs.459642 Unigene Cluster products with high affinity but exhibit low or no antigenicity in the patient.

Fully human monoclonal antibodies of the invention can be generated using cloning technologies employing large human Ig gene combinatorial libraries (i.e., phage display) (Griffiths and Hoogenboom, Building an in vitro immune system: human antibodies from phage display libraries. In: Protein Engineering of Antibody Molecules for Prophylactic and Therapeutic Applications in Man, Clark, M. (Ed.), Nottingham Academic, pp 45-64 (1993); Burton and Barbas, Human Antibodies from combinatorial libraries. Id., pp 65-82). Fully human monoclonal antibodies of the invention can also be produced using transgenic mice engineered to contain human immunoglobulin gene loci (see also, Jakobovits, Exp. Opin. Invest. Drugs 7(4): 607-614 (1998); U.S. Pat. Nos. 6,162,963 issued 19 Dec. 2000; 6,150,584 issued 12 Nov. 2000; and 6,114,598 issued 5 Sep. 2000).

Anti-idiotypic antibodies are also contemplated in the invention. Anti-idiotypic antibodies of the invention can be used to induce an immune response to CSCs. The generation of anti-idiotypic antibodies is well known in the art; this methodology can readily be adapted to generate anti-idiotypic anti-protein of Hs.459642 Unigene Cluster product antibodies that mimic a Hs.459642 Unigene Cluster product epitope [see, for example, Wagner et al., Hybridoma 16:33-40 (1997); Foon et al., J. Clin. Invest. 96:334-342 (1995); Herlyn et al., Cancer Immunol. Immunother. 43:65-76 (1996)]. Anti-idiotypic antibodies can be used to further enhance cancer treatments as described herein.

Antibodies of the invention that treat cancers include those that initiate a potent immune response against the tumor or those that are directly cytotoxic. In this regard, antibodies of the invention can elicit tumor cell lysis by either complement-mediated or antibody-dependent cell cytotoxicity (ADCC) mechanisms, both of which require an intact Fc portion of the immunoglobulin molecule for interaction with effector cell Fc receptor sites on complement proteins. Mechanisms by which directly cytotoxic antibodies act include: inhibition of cell growth, modulation of cellular differentiation, modulation of tumor angiogenesis factor profiles, and the induction of apoptosis. The mechanism(s) by which a particular antibody of the invention exerts an anti-tumor effect can be evaluated using any number of in vitro assays that evaluate cell death such as ADCC, ADMMC, complement-mediated cell lysis, and so forth, as is generally known in the art.

Specificity of the anti-Hs.459642 Unigene Cluster product antibody or antibodies can be tested by many techniques known in the art. For example, the specificity may be determined by ELISA. Whole cells isolated from the patient are used to coat the wells of a multi-well plate, using methods known in the art. Wells are coated with Hs.459642 Unigene Cluster product. Anti-Hs.459642 Unigene Cluster product antibodies are added, and reactivity with Hs.459642 Unigene Cluster products is determined by antibody binding affinity. Other means of determining specificity, well known to those of skill in the art, include FACS analysis and immunochemistry.

Antibodies of the invention can be introduced into a patient such that the antibody binds to a Hs.459642 Unigene Cluster product within a tumor and mediates destruction and/or inhibits the growth of the CSC and other tumor cells. Mechanisms by which such antibodies exert a therapeutic effect can include complement-mediated cytolysis, antibody-dependent cellular cytotoxicity, modulation of the physiological function of proteins of the invention, inhibition of Ca⁺ binding or Ca²⁺ uptake, modulation of tumor cell differentiation, alteration of Ca²⁺ intracellular signaling pathways, and/or apoptosis. An immune response generated against Hs.459642 Unigene Cluster products can lead to, for example, cancer cell death, or reduction in or prevention of, cancer cell proliferation.

In a preferred embodiment, one or more immunostimulants will be administered to the patient in addition to the anti-Hs.459642 Unigene Cluster product antibodies of this invention. An immunostimulant refers to any substance that enhances or potentiates an immune response (antibody and/or cell-mediated) to an antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Upon administration of an antibody composition in accordance with the invention, via injection, aerosol, oral, transdermal, transmucosal, intrapleural, intrathecal, or other suitable routes, the immune system of the patient responds by producing large amounts of immune cells specific for the patient's CSCs. Consequently, the patient becomes immune-sensitized to such cancer cells, or the patient derives at least some therapeutic benefit.

Further contemplated in this disclosure are methods of treating patients with the antibodies of the invention conjugated to a cytotoxic agent. It is routine to conjugate antibodies to cytotoxic agents [see, Sievers et al., Blood 93:11 3678-3684 (1999)]. When cytotoxic and/or therapeutic agents are delivered directly to cells, such as by conjugating them to antibodies specific for a molecule expressed by that cell, the cytotoxic agent will exert its known biological effect (i.e. cytotoxicity) on those cells. For example, antibodies can be conjugated to a toxin or radioisotope, such as the conjugation of calicheamicin or a maytansinoid or Y⁹¹ or I¹³¹ to an antibody.

An initial antibody loading dose of approximately 4 mg/kg patient body weight IV, followed by weekly doses of about 2 mg/kg IV of the antibody preparation represents an acceptable dosing regimen. Preferably, the initial loading dose is administered as a 90 minute or longer infusion. The periodic maintenance dose is administered as a 30 minute or longer infusion, provided the initial dose was well tolerated. As appreciated by those of skill in the art, various factors can influence the ideal dose regimen in a particular case. Such factors include, for example, the binding affinity and half life of the antibody or antibodies used, the degree of expression of the protein of the invention in the patient, the extent of circulating shed Hs.459642 Unigene Cluster product, the desired steady-state antibody concentration level, frequency of treatment, and the influence of chemotherapeutic or other agents used in combination with the treatment method of the invention, as well as the health status of a particular patient.

Antibody formulations of the invention are administered via any route capable of delivering the antibodies to a cancer cell. Routes of administration include, but are not limited to, intravenous, intraperitoneal, intramuscular, intratumor, intradermal, and the like. Treatment generally involves repeated administration of an antibody preparation of the invention, via an acceptable route of administration such as intravenous injection (IV), typically at a dose in the range of about 0.1, 0.2, 0.3, 0.4, 0.5, 0.6, 0.7, 0.8, 0.9, 1, 2, 3, 4, 5, 6, 7, 8, 9, 10, 15, 20, or 25 mg/kg body weight. In general, doses in the range of 10-1000 mg antibodies per week are effective and well tolerated.

Carriers that can be used with vaccines of the invention are well known in the art, and include, e.g., thyroglobulin, albumins such as human serum albumin, tetanus toxoid, polyamino acids such as poly L-lysine, poly L-glutamic acid, influenza, hepatitis B virus core protein, and the like. The vaccines can contain a physiologically tolerable (i.e., acceptable) diluent such as water, or saline, preferably phosphate buffered saline.

This disclosure also provides oligonucleotide inhibitors, including but not limited to antisense oligonucleotides, RNAi, dsRNA, siRNA and ribozymes. Such antisense oligonucleotides antagonize products of the Hs.459642 Unigene Cluster to inhibit proliferation, differentiation or development of stem cells including those involved in cancer. In a preferred example, the antisense oligonucleotides are directed towards CACNA1H.

As used in the specification, “antisense oligonucleotide” refers to a stretch of single-stranded DNA or RNA, usually chemically modified, whose sequence (3′-5′) is complementary to the sense sequence of a molecule of mRNA. Antisense molecules thereby effectively inhibit gene expression by forming RNA/DNA duplexes, and offer a more targeted option for cancer therapy than chemotherapy or radiation. Antisense is believed to work by a variety of mechanisms, including physically blocking the ability of ribosomes to move along the messenger RNA, and hastening the rate at which the mRNA is degraded within the cytosol.

In order to avoid digestion by DNAse, antisense oligonucleotides are often chemically modified. For example, phosphorothioate oligodeoxynucleotides are stabilized to resist nuclease digestion by substituting one of the non-bridging phosphoryl oxygen of DNA with a sulfur moiety. Increased antisense oligonucleotide stability can also be achieved using molecules with 2-methoxyethyl (MOE) substituted backbones as described generally in U.S. Pat. No. 6,451,991, incorporated by reference, and US Published patent application US-2003-0158143-A1. Thus, in the combination and method of the invention, the antisense oligonucleotide is modified to enhance in vivo stability relative to an unmodified oligonucleotide of the same sequence. The modification may be a (2′-O-2-methoxyethyl) modification. The oligonucleotide may have a phosphorothioate backbone throughout, the sugar moieties of nucleotides 1-4 and 18-21 may bear 2′-O-methoxyethyl modifications and the remaining nucleotides may be 2′-deoxynucleotides.

The antisense oligonucleotide may be a 5-10-5 gap-mer methoxyl ethyl modified (MOE) oligonucleotide corresponding to the sequence of a Hs.459642 Unigene Cluster product. The antisense oligonucleotide may be from 10-25 bases in length, or from 15-23 bases in length, or from 18-22 bases in length, or 21 bases in length. In one embodiment, this oligonucleotide has a phosphorothioate backbone throughout.

It is understood in the art that an antisense oligonucleotide need not have 100% identity with the complement of its target sequence in order to be effective. The antisense oligonucleotides in accordance with the present invention, therefore, have a sequence that is at least about 70% identical to the complement of the target sequence. In one embodiment of the present invention, the antisense oligonucleotides have a sequence that is at least about 80% identical to the complement of the target sequence. In other embodiments, they have a sequence that is at least about 90% identical or at least about 95% identical to the complement of the target sequence, allowing for gaps or mismatches of several bases. Identity can be determined, for example, by using the BLASTN program of the University of Wisconsin Computer Group (GCG) software.

The antisense oligonucleotides according to the present invention are typically between 7 and 100 nucleotides in length. In one embodiment, the antisense oligonucleotides comprise from about 7 to about 50 nucleotides, or nucleotide analogues. In another embodiment, the antisense oligonucleotides comprise from about 7 to about 35 nucleotides, or nucleotide analogues. In other embodiments, the antisense oligonucleotides comprise from about 12 to about 35 nucleotides, or nucleotide analogues, and from about 15 to about 25 nucleotides, or nucleotide analogues.

In order for the antisense oligonucleotides of the present invention to function in inhibiting expression of an Hs.459642 product, it is necessary that they demonstrate adequate specificity for the target sequence and do not bind to other nucleic acid sequences in the cell. Therefore, in addition to possessing an appropriate level of sequence identity to the complement of the target sequence, the antisense oligonucleotides of the present invention should not closely resemble other known sequences. The antisense oligonucleotides of the present invention, therefore, should be less than 50% identical to any other mammalian nucleic acid sequence.

Reduction in the amount of Hs.459642 product may also be achieved using RNA interference or “RNAi”. RNAi or double-stranded RNA (dsRNA) directs gene-specific, post-transcriptional silencing in many organisms, including vertebrates. RNA interference mediated by siRNAs is known in the art to play an important role in post-transcriptional gene silencing (Zamore, Nature Struc. Biol., 8:746-750, 2001). In nature, siRNA molecules are typically 21-22 base pairs in length and are generated when long double-stranded RNA molecules are cleaved by the action of an endogenous ribonuclease. RNAi may be effected via directly introducing into the cell, or generating within the cell by introducing into the cell a suitable precursor (e.g. vector, etc.) of such an siRNA or siRNA-like molecule. An siRNA may then associate with other intracellular components to form an RNA-induced silencing complex (RISC). Transfection of mammalian cells with synthetic siRNA molecules having a sequence identical to a portion of a target gene leads to a reduction in the mRNA levels of the target gene (Elbashir et al., Nature, 411:4914498, 2001).

The oligonucleotide inhibitors according to the present invention can be siRNA molecules that are targeted to a gene of interest such that the sequence of the siRNA corresponds to a portion of said gene. RNA molecules used in the present invention generally comprise an RNA portion and some additional portion, for example a deoxyribonucleotide portion. The total number of nucleotides in the RNA molecule is suitably less than 49 in order to be effective mediators of RNAi. In preferred RNA molecules, the number of nucleotides is 16 to 29, more preferably 18 to 23, and most preferably 21-23. In certain embodiments of the invention, the siRNA or siRNA-like molecule is less than about 30 nucleotides in length. In a further embodiment, the siRNA or siRNA-like molecules are about 21-23 nucleotides in length. In an embodiment, siRNA or siRNA-like molecules comprise and 19-21 bp duplex portion, each strand having a 2 nucleotide 3′ overhang. In certain embodiments of the invention, the siRNA or siRNA-like molecule is substantially identical to a Hs.459642 Unigene Cluster product—encoding nucleic acid or a fragment or variant thereof.

The double-stranded siRNA molecules can further comprise poly-T or poly-U overhangs at the 3′ and 5′ ends to minimise RNase-mediated degradation of the molecules. Typically, the overhangs at the 3′ and 5′ ends comprise two thymidine or two uridine residues. Design and construction of siRNA molecules is known in the art (see, for example, Elbashir, et al, Nature, 411:494498, 2001; Bitko and Barik, BMC Microbiol., 1:34, 2001). In addition, kits that provide a rapid and efficient means of constructing siRNA molecules by in vitro transcription are also commercially available (Ambion, Austin, Tex.; New England Biolabs, Beverly, Mass.) and may be used to construct the siRNA molecules of to the present invention.

The present invention further contemplates ribozyme oligonucleotide modulators that specifically target mRNA encoding a protein of interest. Ribozymes are RNA molecules having an enzymatic activity that enables the ribozyme to repeatedly cleave other separate RNA molecules in a nucleotide-sequence specific manner. Such enzymatic RNA molecules can be targeted to virtually any mRNA transcript, and efficient cleavage can be achieved in vitro (Kim et al., Proc. Natl. Acad. Sci. USA, 84:8788, 1987; Haseloff and Gerlach, Nature, 334:585, 1988; Cech, JAMA, 260:3030, 1988; Jefferies et al., Nucleic Acids Res., 17:1371, 1989).

Typically, a ribozyme comprises two portions held in close proximity: an mRNA binding portion having a sequence complementary to the target mRNA sequence, and a catalytic portion which acts to cleave the target mRNA. A ribozyme acts by first recognising and binding a target mRNA by complementary base-pairing through the target mRNA binding portion of the ribozyme. Once it is specifically bound to its target, the ribozyme catalyses cleavage of the target mRNA. Such strategic cleavage destroys the ability of a target mRNA to direct synthesis of an encoded protein. Having bound and cleaved its mRNA target, the ribozyme is released and can repeatedly bind and cleave new target mRNA molecules.

The present invention also provides small molecule inhibitors of a product of the Hs.459642 Unigene Cluster, including peptides, oligonucleotides and synthesised and naturally occurring organic and inorganic molecules. As used herein, a small molecule is defined as a molecule of less than 1200 Daltons, preferably less than 1000 Daltons, or preferably less than 800 Daltons. In a particular example, the small molecule inhibitor inhibits T-type calcium channels including CACNA1H. Known T-type calcium channel inhibitory compounds/small molecules include mibefradil, diltiazem, nifedipine, nitrendipine, nimodipine, niludipine, niguldipine, nicardipine, nisoldipine, amlodipine, felodipine, isradipine, ryosidine, gallopamil, verapamil, tiapamil, pimozide, thioridazine, mibefradil, NNC 55-0396, TTL-1177, anandamide, benzazepine derivatives, and pharmaceutically acceptable salts thereof. Other T-type calcium channel-specific antagonists include 1,3-dioxoisoindole derivatives as disclosed in U.S. Patent No. 7,319,098.

The invention further provides pharmacologic compositions that antagonize products of the Hs.459642 Unigene Cluster to inhibit proliferation, differentiation or development of stem cells including those involved in cancer. The compositions can act by inducing cytotoxicity or by inducing cell death. The compositions can include any of the above-identified inhibitors or antagonists of a product of the Hs.459642 Unigene Cluster, including polyclonal or monoclonal inhibitory antibodies, fully or partially humanized inhibitory antibodies, antisense oligonucleotides, small molecule inhibitors, or any other inhibitor or antagonist disclosed herein. In a particular example, the inhibitor or antagonist, in particular a polyclonal or monoclonal inhibitory antibody, a fully or partially humanized inhibitory antibody, an RNAi, or small molecule inhibitor, inhibits or antagonizes CACNA1H.

A preferred oral dosage form, such as tablets or capsules, can contain the Hs.459642 product inhibitor in an amount of from about 0.1 to about 500 mg, preferably from about 125 to about 200 mg, and more preferably from about 25 to about 150 mg. For parenteral administration, the Hs.459642 product inhibitor can be employed in an amount within the range of from about 0.005 mg/kg to about 10 mg/kg and preferably from about 0.01 mg/kg to about 1 mg/kg.

The pharmacologic compositions and methods of treatment of the invention are further provided in combination with other therapeutic agents to inhibit proliferation, differentiation or development of stem cells, including those involved in cancer. This combination can be administered simultaneously or sequentially. The prophylactically and/or therapeutically effective amount or regimen of an inhibitor of a product of the Hs.459642 Unigene Cluster, particularly an inhibitor of CACNA1H, can be administered herein in combination with one or more additional therapies.

For example, inhibitors of a product of the Hs.459642 Unigene Cluster, particularly an inhibitor of CACNA1H, can be administered in combination with one or more additional cancer therapeutic agents or anti-cancer agents. The terms “cancer therapeutic agent” and “anti-cancer agent” refer to any substance that inhibits or prevents the function, expression, or activity of cells and/or causes destruction of cells. The term is intended to include radioactive isotopes, chemotherapeutic agents, and toxins such as small molecule toxins or enzymatically active toxins of bacterial, fungal, plant or animal origin, including fragments and/or variants thereof. Examples of cytotoxic agents include, but are not limited to maytansinoids, yttrium, bismuth, ricin, ricin A-chain, doxorubicin, daunorubicin, taxol, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, sapaonaria officinalis inhibitor, and glucocorticoid and other chemotherapeutic agents, as well as radioisotopes. In a preferred example, the additional anti-cancer agent is not an inhibitor or antagonist of a product of the Hs.459642 Unigene Cluster, in particular the additional anti-cancer agent is not an inhibitor of CACNA1H.

For example, combination therapy as herein provided by the invention includes treatment with the compositions of the invention in combination with additional treatments that target cancer cells and cancer stem cells. Cancer cells, and cancer stem cells, may be inhibited by antagonists of proliferative signaling pathways such as JAK/STAT, WNT, or p53; by telomerase inhibitors; and by therapeutics targeting cancer stem cell markers such as CD34 (leukemic CSCs), CD138 (myeloma CSCs), and/or CD44 (breast cancer CSCs).

In a preferred example, the compositions of the invention are provided as an “interlaced therapy” in combination with another anti-cancer therapy. In such interlaced therapy, a composition comprising an inhibitor of a product of the Hs.459642 Unigene Cluster, particularly an inhibitor of CACNA1H, is administered to a patient for a period of time, for example, the composition is administered over a period of 1, 3, 5, 7, 14, or 21 or more days, or over 1, 2, or more months. Once the term of administration is completed, the patient is then treated with an additional anti-cancer therapy, according to standard therapeutic regimen for that anti-cancer therapy. In this way, inhibition of the product of the Hs.459642 Unigene Cluster, particularly inhibition of CACNA1H, precedes the additional anti-cancer therapy, and increases or improves the effectiveness of the additional anti-cancer therapy.

The inhibitor of a product of the Hs.459642 Unigene Cluster, particularly an inhibitor of CACNA1H, and the one or more additional anti-cancer therapies can be administered separately, simultaneously, or sequentially, or in any manner best suited for tolerance by the patient. The combination of agents may be administered to a subject by the same or different routes of administration. In alternative embodiments, two or more prophylactic or therapeutic agents are administered in a single composition.

A therapeutically effective amount or regimen of a composition of the invention can be administered to subjects that will, are or have undergone radiation therapy, chemotherapy, hormonal therapy and/or biological therapy including immunotherapy and/or targeted therapy, as well as those who have undergone surgery.

The dosages of the one or more additional anti-cancer agents used in the combination therapy may be lower than those which have been or are currently being used to prevent, treat, and/or manage cancer in the patient. The recommended dosages of the one or more additional therapies currently used for the prevention, treatment, and/or management of cancer can be obtained from any reference in the art including, but not limited to, Hardman et al., eds., Goodman & Gilman's The Pharmacological Basis Of Therapeutics, 10th ed, Mc-Graw-Hill, N.Y., 2001; and Physician's Desk Reference (60^{th} ed., 2006), which are incorporated herein by reference in their entirety.

Thus, for example, a satisfactory result may be obtained employing the Hs.459642 product inhibitor in an amount for oral dosage within the range of from about 0.01 mg/kg to about 100 mg/kg and preferably from about 0.1 mg/kg to about 25 mg/kg in combination with the additional anti-cancer therapeutic agent in an amount within the range of from about 0.01 mg/kg to about 100 mg/kg and preferably from about 0.1 mg/kg to about 25 mg/kg with the Hs.459642 product inhibitor and the additional anti-cancer therapeutic agent being employed together in the same oral dosage form or in separate oral dosage forms taken at the same time.

In one form of treatment, a therapeutically effective amount or regimen of a composition of the invention is administered to a subject that is undergoing or has undergone surgery to remove a tumor, cancer cells or neoplasm. In a specific application, a therapeutically effective amount or regimen of a composition of the invention is administered to a subject concurrently or following surgery to remove a tumor, cancer cells or neoplasm. In another specific application, a therapeutically effective amount or regimen of a composition of the invention is administered to a subject before surgery to remove a tumor or neoplasm and can additionally be administered during and/or after surgery.

In certain embodiments, a therapeutically effective amount or regimen of a composition of the invention is administered to a subject who has failed or is refractory to one or more therapies. A cancer that is “refractory” to a therapy means that at least some significant portion of the cancer cells are not killed or their cell division is not arrested in response to the therapy. The determination of whether the cancer cells are refractory can be made either in vivo or in vitro by any method known in the art for assaying the effect of a therapy on cancer cells, using the art-accepted meanings of refractory in such a context.

A therapeutically effective amount or regimen of a composition of the invention can be administered to patients with increased levels of the cytokine IL-6, which has been associated with the development of cancer cell resistance to different therapeutic regimens, such as chemotherapy and hormonal therapy.

A composition of the invention may comprise one or more Hs.459642 product inhibitors in combination, including any combination of antibody inhibitors, oligonucleotide inhibitors, small molecule inhibitors, or calcium channel blockers, as provided herein. The invention further provides any combination of Hs.459642 product inhibitors in combination with any other anti-cancer therapies as described herein.

The amount of cancer stem cells can be monitored using standard techniques known to one of skill in the art. Cancer stem cells can be monitored by, e.g., obtaining a sample, such as a tissue/tumor sample, blood sample or a bone marrow sample, from a subject and detecting cancer stem cells in the sample. The amount of cancer stem cells in a sample (which may be expressed as percentages of, e.g., overall cells or overall cancer cells) can be assessed by detecting the expression of Hs.459642 product on cancer stem cells. Techniques known to those skilled in the art can be used for measuring these activities. Hs.459642 product expression can be assayed, for example, by immunoassays including, but not limited to, western blots, immunohistochemistry, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, immunofluorescence, protein A immunoassays, flow cytometry, and FACS analysis. In such circumstances, the amount of cancer stem cells in a test sample from a subject may be determined by comparing the results to the amount of stem cells in a reference sample (e.g., a sample from a subject who has no detectable cancer) or to a predetermined reference range, or to the CSCs in the patient at an earlier time point (e.g. prior to, or during therapy).

This disclosure provides a method for detection of stem cells and cancer stem cells by administering an agent or marker that specifically binds to a product of the Hs.459642 Unigene Cluster to an individual or a biological sample obtained from the individual.

This disclosure further provides a method for detection of stem cells and cancer stem cells by administering an antibody directed to a product of the Hs.459642 Unigene Cluster to an individual or a biological sample obtained from the individual. For example, detection of the Hs.459642 Unigene Cluster product-specific antibody within a tumor identifies CSCs within the tumor.

The aforementioned markers can be used to identify cancer stem cells using conventional methods such as immunohistochemistry or cell sorting. Cancer stem cells can be identified essentially using the cell sorting methods described by Al-Hajj et al., Proc. Natl. Acad. Sci. U.S.A. 100: 3984-3983, 2003). The present invention provides an adaptation of this method such that cancer stem cells that express a product of the Hs.459642 Unigene Cluster can be identified using anti- Hs.459642 product antibodies.

In one embodiment, identification of cancer stem cells can be performed by flow cytometry using standard cell sorting procedures. For example, cells obtained from patient effusions or biopsies using conventional techniques may be processed by first ficolling the fluid (typically 500 ml-2 L) to remove debris and red blood cell contamination. Gating can also be carried out to distinguish over blood cells. Flow cytometric staining for cancer stem cell phenotypic analysis can identify “lineage negative” cells. For FACS analysis, FITC-labeled anti-Hs.459642 product antibodies can be used to assay cells from cancer patient tumor tissue.

Anti-Hs.459642 product antibodies of the invention can be used for a variety of diagnostic assays, imaging methodologies, and therapeutic methods in the management of cancers. For example, efficacy of the present method in inhibiting the growth of, or eliminating cancer stem cells in an individual could be ascertained by analysis of samples obtained from the individual before and after treatment, such as by analysis of pre- and post-treatment biopsies, immunohistochemical analysis, or cell sorting analysis to determine the presence of cancer stem cells that express Hs.459642 product. The candidate compound may be isolated or unisolated, pure, partially purified, or in the form of a crude mixture; for example, it may be in the form of a cell, a lysate or extract derived from a cell, or a molecule derived from a cell. Where the candidate compound is present in a composition that comprises more than one molecular entity, it is contemplated that the composition may be tested as is and/or may optionally be fractionated by a suitable procedure and the fractionated sample tested using the method of the invention or another method to identify a particular fraction or component of the composition that acts as an inhibitor of a product of the Hs.459642 Unigene Cluster, particularly an inhibitor of CACNA1H. It is further contemplated that sub-fractions of test compositions may be re-fractionated and assayed repeatedly using the methods of the invention with the ultimate goal of excluding inactive components from the sub-combination identified as an inhibitor of a product of the Hs.459642 Unigene Cluster. Intervening steps of compound isolation, purification and/or characterisation may be included as needed or appropriate.

Candidate compounds can be obtained in the form of large libraries of synthetic or natural compounds. Numerous means are currently used for random and directed synthesis of saccharide, peptide, and nucleic acid based compounds and are well-known in the art. Synthetic compound libraries are commercially available from a number of companies including Maybridge Chemical Co. (Trevillet, Cornwall, UK), Comgenex (Preton, N.J.), Brandon Associates (Merrimack, N.H.), Microsource (New Milford, Conn.), and Aldrich (Milwaukee, Wis.). Combinatorial libraries are also available or can be prepared according to standard procedures. Alternatively, libraries of natural compounds in the form of bacterial, fungal, plant, and animal extracts are available from, for example, Pan Laboratories (Bothell, Wash.) or MycoSearch (North Carolina), or can be readily produced. Additionally, natural and synthetically produced libraries and compounds are readily modified through conventional chemical, physical, and biochemical means.

The application is further described by the following non-limiting examples.

### EXAMPLE 1

In order to determine the effect of inhibition of a product of the Hs.459642 Unigene Cluster on cancer stem cells, the effect of two inhibitors of T-type calcium channels was studied to determine if CACNA1H inhibition sensitizes cancer cells to further anti-cancer treatment. Each inhibitor was administered in vitro in a glioma stem cell line comprising glioblastoma multiforme progenitor cells, followed by treatment with an anti-cancer agent. Subsequent to the combination treatment, with the anti-cancer agent administered sequentially to the CACNA1H inhibitor, fluorescence intensity indicative of cell proliferation was analyzed, with greater intensity indicating increased cellular proliferation.

Effect of mibefradil (Mi) on sensitizing glioma stem cells (GSC) to Temozolomide (TMZ). The GSCs were seeded in 24 well plates as 50,000 cells/well overnight. They were grown in serum-free neurobasal media (Invitrogen), supplemented with human recombinant EGF, bFGF (R&D systems) at 37° C. in a humidified, 5% CO₂ environment. Cells were treated with the T type calcium channel blockers mibefradil at a final concentration of 30 nM or TTL-1177 at a final concentration of 3 μM for 24 hrs, washed and then treated with anti-cancer agent Temozolomide at a final concentration of 5 μM for 24 hrs. The reagent alamarBlue® (Invitrogen) was added to each well to arrive at a final concentration of 10% alamarBlue® in each well. Plates were incubated for 1-2 hours and 100 μl of supernatant was transferred to a 96 well plate for fluorescence reading with an excitation wavelength of 540 nm and emission measured at 590 nm. GBM, glioblastoma multiforme. Mi, mibefradil (Mi). TMZ, Temozolomide. GSC, glioma stem cells.

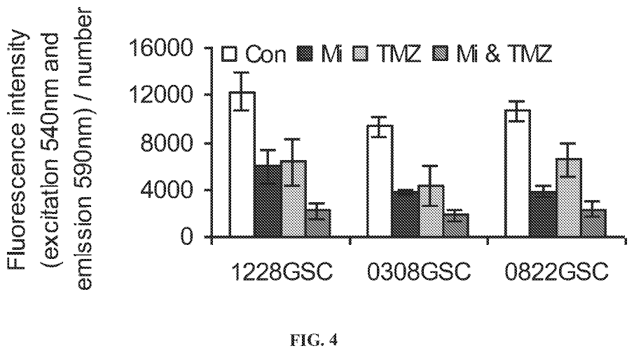

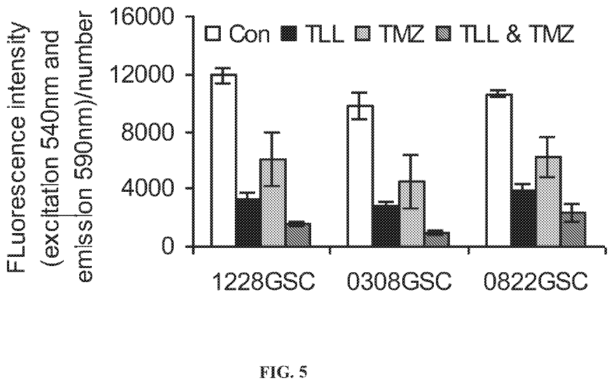

As seen in FIG. 4, fluorescence intensity was dramatically reduced in cells administered combination therapy that included Mi followed by TMZ.

Effect of TTL-1177 (TTL) on sensitizing GSCs to Temozolomide (TMZ). The GSCs were seeded using the same method as in FIG. 4. Cells were treated with the T type calcium channel blocker TTL-1177 (30 μM) for 24 hrs and then treated with Temozolomide (5 μM) for 24 hrs. The cells were tested using an alamarBlue® assay as described above. GBM, glioblastoma multiforme. TTL, TTL-1177. GSC, glioma stem cells.

As shown in FIG. 5, fluorescence intensity was dramatically reduced in cells administered combination therapy that included TTL-1177 followed by TMZ.

### EXAMPLE 2

A patient has lung cancer characterized by a tumor evident in the patient's lung. Immunohistochemical analysis of cancer cells removed from the patient by biopsy of the lung tumor is performed. An anti-Hs.459642 product antibody, specifically an antibody that binds to CACNA1H, is applied to the patient biopsy sample under conditions suitable to allow binding of the antibody to CACNA1H antigen present in the sample. Analysis of the post-treated sample reveals a subpopulation of CSCs within the tumor as identified by the presence of CACNA1H-positive cells in the sample.

The patient is treated with an interlaced therapy regimen that includes treatment with a CACNA1H-specific antibody, or RNA antisense oligonucleotides inhibitor complementary to an RNA sequence of CACNA1H, followed by treatment with a chemotherapeutic anti-cancer agent.

In response to the combination therapy, the patient's tumor regresses and no metastases form.

The invention has been described with reference to various specific and preferred embodiments and techniques. However, it should be understood that many variations and modifications can be made while remaining within the spirit and scope of the invention. It will be apparent to one of ordinary skill in the art that compositions, methods, devices, device elements, materials, procedures and techniques other than those specifically described herein can be applied to the practice of the invention as broadly disclosed herein without resort to undue experimentation. All art-known functional equivalents of compositions, methods, devices, device elements, materials, procedures and techniques described herein are intended to be encompassed by this invention. Whenever a range is disclosed, all subranges and individual values are encompassed. This invention is not to be limited by the embodiments disclosed, including any exemplified in the specification, which are given by way of example or illustration and not of limitation. The scope of the invention shall be limited only by the claims.

1-22. (canceled)

23. A method for treating a brain tumor in a patient, the method comprising:
(a) targeting cancer stem cells in the brain tumor of a patient by administering to the patient a therapeutically effective amount of a CACNA1H antagonist, wherein the cancer stem cells exhibit expression activity in the Hs.459642 Unigene Cluster, wherein the CACNA1H antagonist is mibefradil or TTL-177; and
(b) administering an additional anti-cancer therapy to the patient, thereby treating the brain tumor.

24. The method of claim 23 wherein the additional anti-cancer therapy is selected from the group consisting of surgery, radiation therapy, and administration of a chemotherapeutic agent.

25. The method of claim 23, wherein the patient is a human.

26. The method of claim 23, wherein the brain cancer is a glioblastoma.

27. The method of claim 23, wherein the antagonist is administered to the patient prior to the additional anti-cancer therapy.

28. The method of claim 23, wherein the antagonist and the additional anti-cancer therapy are administered to the patient simultaneously.

29. The method of claim 23, wherein the antagonist is mibefradil.

30. The method of claim 23, wherein the antagonist is TTL-177.

31. The method of claim 23, wherein the additional anti-cancer therapy is administration of a chemotherapeutic agent, and wherein the chemotherapeutic agent is temozolomide.

32. The method of claim 23, wherein the cancer stem cells are glioma stem cells.

33. The method of claim 23, wherein the cancer is a refractory or recurrent cancer.
